(19) United States
(12) Patent Application Publication
(10) Pub. No.: US 2022/0240772 A1
(43) Pub. Date: Aug. 4, 2022

(54) CORRECTIVE LIGHT FIELD DISPLAY PROFILE MANAGEMENT, COMMUNICATION AND INTEGRATION SYSTEM AND METHOD

(71) Applicant: EVOLUTION OPTIKS LIMITED, Christ Church (BB)

(72) Inventors: Raul Mihali, Westport, CT (US); Jean-François Joly, Victoria (CA); Andre Michel Daniel MERIZZI, Ottawa (CA); Joseph Ivar ETIGSON, Toronto (CA)

(21) Appl. No.: 17/594,699

(22) PCT Filed: May 5, 2020
(86) PCT No.: PCT/US2020/031455

§ 371 Date: Oct. 26, 2021

(30) Foreign Application Priority Data
May 9, 2019 (CA) 3042823
May 9, 2019 (CA) 3042824

(51) Int. Cl. A61B 3/00

(52) U.S. Cl. CPC A61B 3/0025; G16H 10/65; A61B 3/0041; A61B 3/0033

(57) ABSTRACT

Described are various embodiments of a digital vision correction system to at least partially address a user's reduced visual acuity. In some embodiments, the system comprises a user mobile device and a distinct electronic display device comprising a light field display and a network interface operable to interface with the user mobile device to access a vision correction parameter therefrom. In a same or other embodiment, a digital display device is provided to render an input image for viewing by a viewer having reduced visual acuity that varies as a function of a current viewing condition.

### CROSS REFERENCE TO RELATED APPLICATIONS

This application claims priority to Canadian Patent Application No. 3,042,824 filed May 9, 2019 and Canadian Patent Application No. 3,042,823 filed May 9, 2019, the entire disclosure of each of which is hereby incorporated herein by reference.

### FIELD OF THE DISCLOSURE

The present disclosure relates to electronic devices having a graphical display, and, in particular, to a corrective light field display profile management, communication and integration system and method.

### BACKGROUND

Individuals routinely wear corrective lenses to accommodate for reduced vision acuity in consuming images and/or information rendered, for example, on digital displays provided, for example, in day-to-day electronic devices such as smartphones, smart watches, electronic readers, tablets, laptop computers and the like, but also provided as part of vehicular dashboard displays and entertainment systems, to name a few examples. The use of bifocals or progresses corrective lenses is also commonplace for individuals suffering from near and far sightedness.

The operating systems of current electronic devices having graphical displays offer certain “Accessibility” features built into the software of the device to attempt to provide users with reduced vision the ability to read and view content on the electronic device. Specifically, current accessibility options include the ability to invert images, increase the image size, adjust brightness and contrast settings, bold text, view the device display only in grey, and for those with legal blindness, the use of speech technology. These techniques focus on the limited ability of software to manipulate display images through conventional image manipulation, with limited success.

Light field displays using lenslet arrays or parallax barriers have been proposed for correcting such visual aberrations. For a thorough review of Autostereoscopic or light field displays, Halle M. (Halle, M., “Autostereoscopic displays and computer graphics” ACM SIGGRAPH, 31(2), pp. 58-62, 1997) gives an overview of the various ways to build a glasses-free 3D display, including but not limited to parallax barriers, lenticular sheets, microlens arrays, holograms, volumetric displays for example. Moreover, the reader is also directed to another article by Masia et al. (Masia B., Wetzstein G., Didyk P. and Gutierrez, “A survey on computational displays: Pushing the boundaries of optics, computation and perception”, Computer & Graphics 37 (2013), 1012-1038) also provides a good review of computational displays, notably light field displays at section 7.2 and vision correcting light field displays at section 7.4.

A first example of using light field displays to correct visual aberrations has been proposed by Pamplona et al. (PAMPLONA, V., OLIVEIRA, M., ALIAGA, D., AND RASKAR, R.2012. “Tailored displays to compensate for visual aberrations.” ACM Trans. Graph. (SIGGRAPH) 31.). Unfortunately, conventional light field displays as used by Pamplona et al. are subject to a spatio-angular resolution trade-off; that is, an increased angular resolution decreases the spatial resolution. Hence, the viewer sees a sharp image but at the expense of a significantly lower resolution than that of the screen. To mitigate this effect, Huang et al. (see, HUANG, F.-C., AND BARSKY, B. 2011. A framework for aberration compensated displays. Tech. Rep. UCB/EECS-2011-162, University of California, Berkeley, December; and HUANG, F.-C., LANMAN, D., BARSKY, B. A., AND RASKAR, R. 2012. Correcting for optical aberrations using multi layer displays. ACM Trans. Graph. (SiGGRAPH Asia) 31, 6, 185:1-185:12. proposed the use of multilayer display designs together with prefiltering. The combination of prefiltering and these particular optical setups, however, significantly reduces the contrast of the resulting image.

Moreover, in U.S. Patent Application Publication No. 2016/0042501 and Fu-Chung Huang, Gordon Wetzstein, Brian A. Barsky, and Ramesh Raskar. “Eyeglasses-free Display: Towards Correcting Visual Aberrations with Computational Light Field Displays”. ACM Transaction on Graphics, xx:0, August 2014, the entire contents of each of which are hereby incorporated herein by reference, the combination of viewer-adaptive pre-filtering with off-the-shelf parallax barriers has been proposed to increase contrast and resolution, at the expense however, of computation time and power.

Another example includes the display of Wetzstein et al. (Wetzstein, G. et al., “Tensor Displays: Compressive Light Field Synthesis using Multilayer Displays with Directional B acklighting”, https://web.media.mit.edu/˜gordonw/TensorDisplays/Tensor Displays.pdf) which disclose a glasses-free 3D display comprising a stack of time-multiplexed, light-attenuating layers illuminated by uniform or directional backlighting. However, the layered architecture may cause a range of artefacts including Moiré effects, color-channel crosstalk, interreflections, and dimming due to the layered color filter array. Similarly, Agus et al. (AGUS M. et al., “GPU Accelerated Direct Volume Rendering on an Interactive Light Field Display”, EUROGRAPHICS 2008, Volume 27, Number 2, 2008) disclose a GPU accelerated volume ray casting system interactively driving a multi-user light field display. The display, produced by the Holographika company, uses an array of specially arranged array of projectors and a holographic screen to provide glass-free 3D images. However, the display only provides a parallax effect in the horizontal orientation as having parallax in both vertical and horizontal orientations would be too computationally intensive. Finally, the FOVI3D company (http://on-demand.gputechconf.com/gtc/2018/presentation/s8461-extreme-multi-view-rendering-for-light-field-displays .pdf) provides light field displays wherein the rendering pipeline is a replacement for OpenGL which transports a section of the 3D geometry for further processing within the display itself.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a corrective light field display, system and method, that overcome some of the drawbacks of known techniques, or at least, provide a useful alternative thereto. Some aspects of disclosure provide embodiments of such systems and methods. For example, some embodiments provide for a variable corrective light field profile, and/or various profile management, communication and integration functions.

For instance, while some corrective systems and methods as described herein may be implemented to be configured or calibrated specifically for a user's decreased visual acuity, whereby the user enters a set of vision correction parameters into the device controlling the light field display so that the light field display may be able to render an image which would, at least partially, compensate for the user's reduced visual acuity, further methods and systems are described herein so to improve and/or at least partially automate user operability of such systems and methods. Namely, a more efficient method is needed to manage the configuration/calibration of these light field displays to a user's specific set of vision correction parameters, and/or to facilitate the communication and selective distribution of such digital parameters and characteristics between devices.

In accordance with one aspect, there is provided a digital vision correction system to at least partially address a user's reduced visual acuity, the system comprising: a user mobile device comprising: a processing unit; a digital data storage to store a digital vision correction parameter associated with the user's reduced visual acuity; and a wireless network interface; and a distinct electronic display device comprising: a light field display operable to render digital content; a network interface operable to interface with said user mobile device to access said vision correction parameter; and a processing unit, communicatively linked to said light field display and network interface, and operable on pixel data associated with said digital content to adjust a rendering thereof via said light field display as a function of said digital vision correction parameter so to at least partially address the user's reduced visual acuity.

In one embodiment, the system comprises a plurality said distinct electronic display device, each operable to respectively interface with said user mobile device to access said digital vision correction parameter and thereby output vision-corrected digital content.

In one embodiment, each said distinct electronic display device is further operable to automatically delete said vision correction parameter therefrom upon termination of a given user's interaction therewith, and access a distinct vision correction parameter for a distinct user upon interfacing with a distinct user mobile device.

In one embodiment, the distinct electronic display device comprises an onboard vehicular data processing device, and wherein said vision correction parameter is accessed from said user mobile device upon wirelessly pairing said user mobile device with said onboard vehicular data processing device.

In one embodiment, the distinct electronic display device comprises an electronic kiosk, and wherein said vision correction parameter is accessed from said user mobile device upon wirelessly interfacing said user mobile device with said electronic kiosk.

In one embodiment, the communication interface comprises at least one of a Bluetooth™ interface or a Near Field Communication (NFC) interface.

In one embodiment, the vision correction parameter is entered or derived from a manual user input.

In one embodiment, the vision correction parameter is entered or derived from a network-interfacing connection to an eye care specialist terminal.

In one embodiment, the user mobile device comprises a light field enabled display operable to render vision-corrected digital content, and wherein said vision correction parameter is dynamically adjusted via a graphical interface rendered on said user mobile device.

In one embodiment, the user mobile device comprises one of a mobile communication device, an electronic key, an electronic key fob, a digital identification card, and/or a wearable device.

In accordance with another aspect, there is provided a digitally implemented vision correction method for adjusting digital content to be rendered by an electronic display device in accordance with distinct digital user profiles each having a respective reduced visual acuity associated therewith, the method comprising, for each given digital user profile: wirelessly interfacing the electronic display device having a light field display with a user mobile device to provide wireless access thereto to a digital user-specific vision correction parameter stored thereon or in association therewith and associated with the respective reduced visual acuity; processing pixel data associated with the digital content to adjust a rendering thereof via said light field display as a function of said digital vision correction parameter; and rendering said adjusted pixel data via said light field display to at partially address the respective reduced visual acuity.

In one embodiment, the method further comprises digitally storing said digital user-specific vision correction parameter on said user mobile device via a communication interface between said user mobile device and an eye care specialist system.

In one embodiment, the method further comprises digitally defining said digital user-specific vision correction parameter on said user mobile device via an onboard vision correction adjustment application executed on said mobile device to be interactively operated by the user in setting said digital user-specific vision correction parameter.

In one embodiment, the method further comprises digitally storing said digital user-specific vision correction parameter on said user mobile device via a communication interface between said user mobile device and a distinct digital vision correction testing device operating a vision correction adjustment application thereon to be interactively operated by the user in setting said digital user-specific vision correction parameter.

In one embodiment, the wirelessly interfacing comprises automatically pairing or wirelessly interfacing said user mobile device upon said user mobile device being in the vicinity of said electronic display device.

In one embodiment, the user mobile device comprises a smartphone, a tablet, a laptop, a smart key, a key fob or an identification card.

In one embodiment, the wirelessly interfacing comprises securely transferring said digital user-specific vision correction parameter.

In one embodiment, the digital user-specific vision correction parameter is securely transferred via a secure digital token.

In one embodiment, the method further comprises, upon termination of said interfacing, deleting said user-specific vision correction parameter from said electronic display device.

Furthermore, as a user's visual acuity, in some examples, may not be static but may rather change as a function of different viewing conditions (e.g. time of day, light level, viewing time, etc.), there is a need to have systems and methods for optimizing/adjusting the corrective power of a light field display as a function of different viewing conditions.

In accordance with one aspect, there is provided a digital display device to render an input image for viewing by a viewer having reduced visual acuity that varies as a function of a current viewing condition, the device comprising: a digital light field display comprising an array of pixels to render a pixelated image accordingly, and an array of light field shaping elements disposed relative to said array of pixels to shape a light field emanating therefrom and thereby at least partially govern a projection thereof toward the viewer; and a hardware processor operable on pixel data for the input image, as a function of a variable visual acuity parameter representative of the viewer's reduced visual acuity, to output adjusted image pixel data to be rendered via said digital display medium and projected through said light field shaping elements so to produce a designated image perception adjustment to at least partially address the viewer's reduced visual acuity; wherein said hardware processor is further operable to automatically monitor the current viewing condition and adjust said variable visual acuity parameter accordingly to accommodate the viewer's reduced visual acuity as it varies as a function of the current viewing condition.

In one embodiment, the viewing condition comprises a time-of-day, and wherein said hardware processor is operable to access said time-of-day to adjust said variable visual acuity parameter accordingly.

In one embodiment, the time-of-day comprises at least designated daytime and night time periods, and wherein said variable visual acuity parameter is set, at least in part, as a function said daytime and night time periods, respectively.

In one embodiment, the viewing condition comprises an elapsed viewing time, and wherein said variable visual acuity parameter is adjusted, at least in part, as a function of said elapsed viewing time so to accommodate a decreasing viewer visual acuity as a function of an increasing elapsed viewing time.

In one embodiment, the viewing condition comprises a viewing content type selected from a designated set of viewing content types.

In one embodiment, the viewing condition comprises ambient lighting.

In one embodiment, the ambient lighting is categorized according to at least two ambient lighting conditions comprising a relatively lower ambient light condition and a relatively higher ambient lighting condition, and wherein said variable visual acuity parameter is adjusted, at least in part, as a function of said ambient lighting condition so to accommodate a decreasing viewer visual acuity as a function of a decrease in ambient lighting.

In one embodiment, the digital display further comprises an optical sensor operable to sense said ambient lighting condition.

In one embodiment, a respective set of variable visual acuity parameters is set as a function of a corresponding set of designated viewing conditions.

In one embodiment, the hardware processor is further operable to render a graphical user interface for manually adjusting said set of variable visual acuity parameters as a function of said current viewing condition such that an initial visual acuity parameter can be manually adjusted by the viewer for said current viewing condition and reset accordingly.

In one embodiment, the hardware processor is further operable to estimate a new visual acuity parameter for a new viewing condition as a function of said set of variable visual acuity parameters.

In accordance with another aspect, there is provided a digitally implemented vision correction method, to be implemented by a digital processor associated with a digital light field display, for adjusting digital content to be rendered by the digital light field display to at least partially accommodate a viewer's reduced visual acuity, the method comprising: automatically identifying a current viewing condition; setting a current vision correction parameter as a function of said automatically identified current viewing condition; and adjusting the digital content as a function of said current vision correction parameter to be rendered accordingly in at least partially accommodating the viewer's reduced visual acuity for said current viewing condition.

In one embodiment, the method further comprises storing respective vision correction parameters associated with the viewer's reduced visual acuity for distinct designated viewing conditions; and wherein said setting comprises setting said current vision correction parameter from said respective vision correction parameters.

In one embodiment, the method further comprises, prior to said storing: receiving as input an initial vision correction parameter associated with the viewer's reduced visual acuity; identifying a given viewing condition; dynamically adjusting said initial vision correction parameter based on a viewer input to accommodate said given viewing condition; storing a given vision correction parameter associated with said given viewing condition based on said dynamically adjusting as one of said respective vision correction parameters; and upon automatically identifying said given viewing condition, setting said current vision correction parameter to said given vision correction parameter for said given viewing condition.

In one embodiment, the dynamically adjusting and said storing are executed in respect of multiple distinct viewing conditions.

In one embodiment, the method further comprises setting said current vision correction parameter as a function of a viewer demographic.

In one embodiment, the current viewing condition comprises at least one of a time-of-day, a daytime period, a night time period, an illuminance, a content time, an elapsed viewing time or a combination thereof.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a schematic diagram of an illustrative corrective light field display profile communication and integration system for configuring/calibrating an external device comprising a light field display for compensating for a user's reduced visual acuity, in accordance with one embodiment;

FIG. 2 is a schematic diagram of an illustrative corrective light field display profile communication and integration system for configuring/calibrating an external device comprising a light field display for compensating for a user's reduced visual acuity, in accordance with another embodiment;

FIG. 3 is a process flow diagram of an illustrative corrective light field display profile communication and integration method, in accordance with one embodiment;

FIG. 4 is a schematic diagram of an exemplary set of vision correction parameters, in accordance with one embodiment;

FIG. 5 is a process flow diagram of an illustrative method for updating a set of vision correction parameters for a corrective light field display profile communication and integration system, as shown for example in FIGS. 1 and 2, in accordance with one embodiment;

FIG. 6 is a process flow diagram of an exemplary set of vision conditions for which vision correction parameters may be automatically adjusted, in accordance with one embodiment;

FIG. 7 is a schematic diagram illustrating a dynamic corrective power management system for light field displays, in accordance with one embodiment; and

FIG. 8 is a process flow diagram of an exemplary time-variable corrective light field display management method using the system of FIG. 7, in accordance with one embodiment.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

The systems and methods described herein provide, in accordance with different embodiments, different examples of a (variable) vision correction system and method for automatically configuring/calibrating a corrective light field display profile, and/or facilitate transfer or communication of profile information related thereto in implementing corrective light field display features and/or functions on different adaptive light field enabled devices so to at least partially compensate for a user's reduced visual acuity.

For example, most people have to interact with various digital displays on a routine or recurring basis, whether it be with their personal smartphone, tablet, TVs, or like personal display devices, while travelling via onboard vehicular displays such as car and/or airplane infotainment systems or similar, or again at various point of sale or public information terminals or kiosks such as venue or event ticketing kiosks, travel document verification or issuing kiosks, automated point of sale kiosks, on-premises consumer product sales, information or assistance kiosks, professional services sign-in kiosks, or the like. Collaborative or distributive workspaces may also involve user's interfacing with different digital displays or terminals on a day-to-day basis. Generally, these displays are configured to render digital and optionally interactive images comprising text, pictures, graphics, videos or other such visual information.

In accordance with some of the herein-described embodiments, traditional displays as noted above can be replaced with light field displays, as described below, so to enable rendering of corrective light field images, or portions thereof that, when executed in accordance with a user's corrective light field profile, can at least partially accommodate for this user's reduced visual acuity. For instance, light-field enabled displays or kiosks as described herein may be operable to render digital content via a selectively controllable light field (e.g. directional light rays, as will be further explained below) and used for example, to compensate for a user's reduced visual acuity while the user is viewing such content, thus, in some implementations, removing the need for the user to put on corrective eyewear or the like. Naturally, any such light field display (as will be explained below) will require access to at least some information about a given user's reduced visual acuity so to properly render an image that at least partially compensates for this user's specific (or range of) visual acuity. In some embodiments, to increase usability and user accommodation, such profile-specific configuration/calibration may be automatically communicated and managed between devices and/or displays, reducing the need for manual operation and/or routine recalibrations.

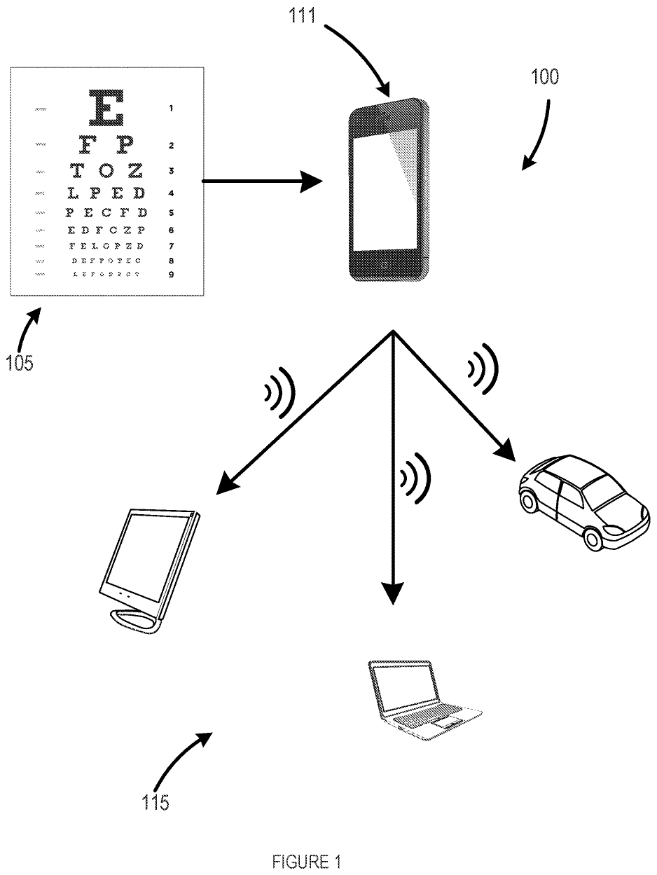

As illustrated in FIG. 1, in some embodiments, the system and method may provide a user the ability of carrying a set of vision correction parameter(s), herein derived from an eye exam 105 for example, on a mobile device 111 operable to communicate/transmit said set of vision correction parameter(s) to any nearby light-field capable display device 115 (as will be discussed below) to configure/calibrate said device to render images that, at least partially, compensate specifically for the user's reduced visual acuity. Thus, in some embodiments, the systems and methods discussed herein provide an efficient means of quickly and securely configuring/calibrating any light field capable display (or just light field display) which avoids the user having to manually enter a set of vision correction parameters into each new light field device he/she interacts with, or again, invoke a local corrective light field calibration process. In some embodiments, a mobile device locally storing or remotely managing access to a user's corrective light field profile can be paired wirelessly via Bluetooth™ and/or transmit corrective light field data via a near-field communication (NFC) protocol, or the like, such that such information can be effortlessly and wirelessly communicated or exchanged with the paired or receiving light field display device to implement appropriate corrective light field operations. Thus, a user may go about his/her day and interact with different displays in a multiplicity of contexts 115 (e.g. dashboard or multimedia display in a vehicle, ticketing booth or monitor, TVs, computer screens, phone/tablets, etc. as will be explained below), all of which, if equipped with a light-field capable display, would be, in some embodiments automatically configured/calibrated upon gaining access to the user's personal corrective light field profile or information via the user's portable device, to automatically compensate for the user's specific reduced vision acuity, and that, without any additional input from the user.

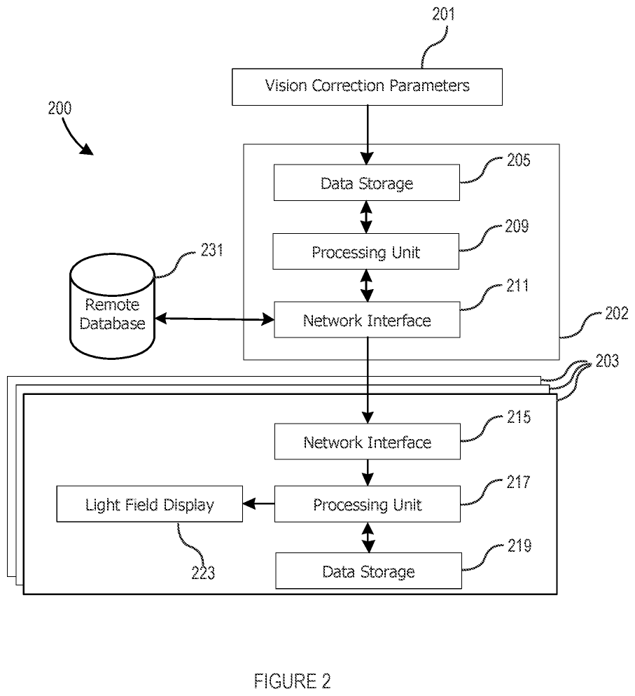

With reference to FIG. 2, and in accordance with one exemplary embodiment, a vision correction management system, generally referred to using the numeral 200, will now be described. In some embodiments, the system 200 may be operable to manage, store and communicate a set of one or more vision correction parameter(s) 201 to a light field display in a secure and easy-to-use fashion. The set of vision correction parameters 201 generally comprise any data representative of the user's reduced visual acuity or condition (as will be further discussed below), but in a form that may be used directly to configure or calibrate a light field rendering algorithm, such as a light field ray tracing algorithm or the like, that, when executed in accordance with these parameters, results in the rendering of images that compensate, at least partially, for the user's reduced vision acuity.

Generally, a mobile device 202 will be used by a given user to locally manage and/or intermediate management of the user's vision correction profile, and may include, as discussed herein, any electronic or mobile device comprising a data storage unit or internal memory 205, and a network interface 211 for storing and communicating, respectively, a set of vision correction parameters 201, or the like, to other devices and/or systems 203 each comprising one or more light field capable displays 223 for which corrective light field rendering is required or desired. Device 202 may include, without limitation, smartphones, laptops, tablets, smartwatches or like wearables, electronic car keys (e.g. smart key or fob), digital ID cards, RFID cards or similar. Internal memory 205 can be any form of electronic storage, including a disk drive, optical drive, read-only memory, random-access memory, or flash memory, to name a few examples.

In some embodiments, mobile device 202 may be communicatively linked to an external database 231 via network interface 211. External database 231 may be used to store and transmit the set of vision correction parameter(s) 201 to the mobile device 202, as will be explained below, or again to relay such information via or upon direction from the mobile device 202, for example.

Generally, and as introduced above, each digital display device or system 203 will general comprise a processing unit 217, a network interface 215 to receive the set of vision correction parameter(s) 201 from the mobile device 202 and/or remote database 231, a data storage unit or internal memory 219 to permanently or temporally store said set of vision correction parameters 201, and a light field display 223. Internal memory 219 can be any form of electronic storage, including a disk drive, optical drive, read-only memory, random-access memory, or flash memory, to name a few examples. Internal memory 219 also generally comprises any data and/or programs needed to properly operate light field display 223 via processing unit 217. Other components of electronic device 203 may optionally include, but are not limited to, one or more rear and/or front-facing camera(s) (e.g. for onboard pupil tracking capabilities), pupil tracking light source, an accelerometer and/or other device positioning/orientation devices capable of determining the tilt and/or orientation of electronic device, or the like.

For example, electronic device 203, or related environment (e.g. within the context of a desktop workstation, vehicular console/dashboard, gaming or e-learning station, multimedia display room, digital kiosk or terminal, etc.) may include further hardware, firmware and/or software components and/or modules to deliver complementary and/or cooperative features, functions and/or services (not shown). For example, a pupil/eye tracking system may be integrally or cooperatively implemented to improve or enhance corrective image rendering by tracking a location of the user's eye(s)/pupil(s) (e.g. both or one, e.g. dominant, eye(s)) and adjusting light field corrections accordingly). For instance, the device may include, integrated therein or interfacing therewith, one or more eye/pupil tracking light sources, such as one or more infrared (IR) or near-IR (NIR) light source(s) to accommodate operation in limited ambient light conditions, leverage retinal retro-reflections, invoke corneal reflection, and/or other such considerations. For instance, different IR/NIR pupil tracking techniques may employ one or more (e.g. arrayed) directed or broad illumination light sources to stimulate retinal retro-reflection and/or corneal reflection in identifying and tracking a pupil location. Other techniques may employ ambient or IR/NIR light-based machine vision and facial recognition techniques to otherwise locate and track the user's eye(s)/pupil(s). To do so, one or more corresponding (e.g. visible, IR/NIR) cameras may be deployed to capture eye/pupil tracking signals that can be processed, using various image/sensor data processing techniques, to map a 3D location of the user's eye(s)/pupil(s). In the context of a mobile device, such as a mobile phone, such eye/pupil tracking hardware/software may be integral to the device, for instance, operating in concert with integrated components such as one or more front facing camera(s), onboard IR/NIR light source(s) and the like. In other user environments, such as in a vehicular environment, eye/pupil tracking hardware may be further distributed within the environment, such as dash, console, ceiling, windshield, mirror or similarly-mounted camera(s), light sources, etc.

Generally, a light field display 223 as discussed herein will comprise a set of image rendering pixels and a light field shaping layer disposed at a preset distance therefrom so to controllably shape or influence a light field emanating therefrom. For instance, each light field shaping layer will be defined by an array of optical elements centered over a corresponding subset of the display's pixel array to optically influence a light field emanating therefrom and thereby govern a projection thereof from the display medium toward the user, for instance, providing some control over how each pixel or pixel group will be viewed by the viewer's eye(s). As will be further detailed below, arrayed optical elements may include, but are not limited to, lenslets, microlenses or other such diffractive optical elements that together form, for example, a lenslet array; pinholes or like apertures or windows that together form, for example, a parallax or like barrier; concentrically patterned barriers, e.g. cut outs and/or windows, such as a to define a Fresnel zone plate or optical sieve, for example, and that together form a diffractive optical barrier (as described, for example, in Applicant's co-pending U.S. application Ser. No. 15/910,908, the entire contents of which are hereby incorporated herein by reference); and/or a combination thereof, such as for example, a lenslet array whose respective lenses or lenslets are partially shadowed or barriered around a periphery thereof so to combine the refractive properties of the lenslet with some of the advantages provided by a pinhole barrier.

In operation, the display 223 will also generally invoke hardware processor 217 operable on image pixel (or subpixel) data for an image to be displayed to output corrected or adjusted image pixel data to be rendered as a function of a stored characteristic of the light field shaping layer (e.g. layer distance from display screen, distance between optical elements (pitch), absolute relative location of each pixel or subpixel to a corresponding optical element, properties of the optical elements (size, diffractive and/or refractive properties, etc.), or other such properties, and a selected vision correction or adjustment parameter related to the user's reduced visual acuity or intended viewing experience. While light field display characteristics will generally remain static for a given implementation (i.e. a given shaping layer will be used and set for each device irrespective of the user), image processing can, in some embodiments, be dynamically adjusted as a function of the user's visual acuity or intended application so to actively adjust a distance of a virtual image plane, or perceived image on the user's retinal plane given a quantified user eye focus or like optical aberration(s), induced upon rendering the corrected/adjusted image pixel data via the static optical layer, for example, or otherwise actively adjust image processing parameters as may be considered, for example, when implementing a viewer-adaptive pre-filtering algorithm or like approach (e.g. compressive light field optimization), so to at least in part govern an image perceived by the user's eye(s) given pixel or subpixel-specific light visible thereby through the layer.

Using a set of vision correction parameter(s) 201, electronic device or system 203 can be configured to render via display 223 a corrected image via the corrective light field layer that accommodates for the user's visual acuity. By adjusting the image correction in accordance with the user's actual predefined, set or selected visual acuity level, different users and visual acuities may be accommodated using a same device configuration. That is, by adjusting corrective image pixel data to dynamically adjust a virtual image distance below/above the display as rendered via the light field layer, different visual acuity levels may be accommodated.

In general, device 203 as considered herein may include, but is not limited to, smartphones, tablets, e-readers, watches, televisions, GPS devices, laptops, desktop computer monitors, televisions, smart televisions, handheld video game consoles and controllers, vehicular dashboard and/or entertainment displays, ticketing or shopping kiosks, point-of-sale (POS) systems, workstations, or the like.

Display 223 can comprise an LCD screen, a monitor, a plasma display panel, an LED or OLED screen, or any other type of digital display defined by a set of pixels for rendering a pixelated image or other like media or information.

Furthermore, electronic device 203 in this example will comprise a light field shaping layer (LFSL) overlaid atop a pixel display thereof and spaced therefrom (e.g. via an integrated or distinct spacer) or other such means as may be readily apparent to the skilled artisan. For the sake of illustration, the following examples will be described within the context of a light field shaping layer defined, at least in part, by a lenslet array comprising an array of microlenses (also interchangeably referred to herein as lenslets) that are each disposed at a distance from a corresponding subset of image rendering pixels in an underlying digital display. It will be appreciated that while a light field shaping layer may be manufactured and disposed as a digital screen overlay, other integrated concepts may also be considered, for example, where light field shaping elements are integrally formed or manufactured within a digital screen's integral components such as a textured or masked glass plate, beam-shaping light sources or like component. Accordingly, each lenslet will predictively shape light emanating from these pixel subsets to at least partially govern light rays being projected toward the user by the display device. As noted above, other light field shaping layers may also be considered herein without departing from the general scope and nature of the present disclosure, whereby light field shaping will be understood by the person of ordinary skill in the art to reference measures by which light, that would otherwise emanate indiscriminately (i.e. isotropically) from each pixel group, is deliberately controlled to define predictable light rays that can be traced between the user and the device's pixels through the shaping layer.

For greater clarity, a light field is generally defined as a vector function that describes the amount of light flowing in every direction through every point in space. In other words, anything that produces or reflects light has an associated light field. The embodiments described herein produce light fields from an object that are not “natural” vector functions one would expect to observe from that object. This gives it the ability to emulate the “natural” light fields of objects that do not physically exist, such as a virtual display located far behind the light field display, which will be referred to now as the ‘virtual image’. As noted in the examples below, in some embodiments, lightfield rendering may be adjusted to effectively generate a virtual image on a virtual image plane that is set at a designated distance from an input user pupil location, for example, so to effectively push back, or move forward, a perceived image relative to the display device in accommodating a user's reduced visual acuity (e.g. minimum or maximum viewing distance). In yet other embodiments, lightfield rendering may rather or alternatively seek to map the input image on a retinal plane of the user, taking into account visual aberrations, so to adaptively adjust rendering of the input image on the display device to produce the mapped effect. Namely, where the unadjusted input image would otherwise typically come into focus in front of or behind the retinal plane (and/or be subject to other optical aberrations), this approach allows to map the intended image on the retinal plane and work therefrom to address designated optical aberrations accordingly. Using this approach, the device may further computationally interpret and compute virtual image distances tending toward infinity, for example, for extreme cases of presbyopia. This approach may also more readily allow, as will be appreciated by the below description, for adaptability to other visual aberrations that may not be as readily modeled using a virtual image and image plane implementation. In both of these examples, and like embodiments, the input image is digitally mapped to an adjusted image plane (e.g. virtual image plane or retinal plane) designated to provide the user with a designated image perception adjustment that at least partially addresses designated visual aberrations.

Different examples of ray tracing and corrective light field shaping processes are provided in Applicant's co-pending U.S. patent application Ser. No. 16/259,845 filed Jan. 28, 2019, the entire contents of which are hereby incorporated herein by reference. While visual aberrations may be addressed using these approaches, other visual effects may also be implemented using similar techniques, and can other techniques be considered herein without departing from the general scope and nature of the present disclosure.

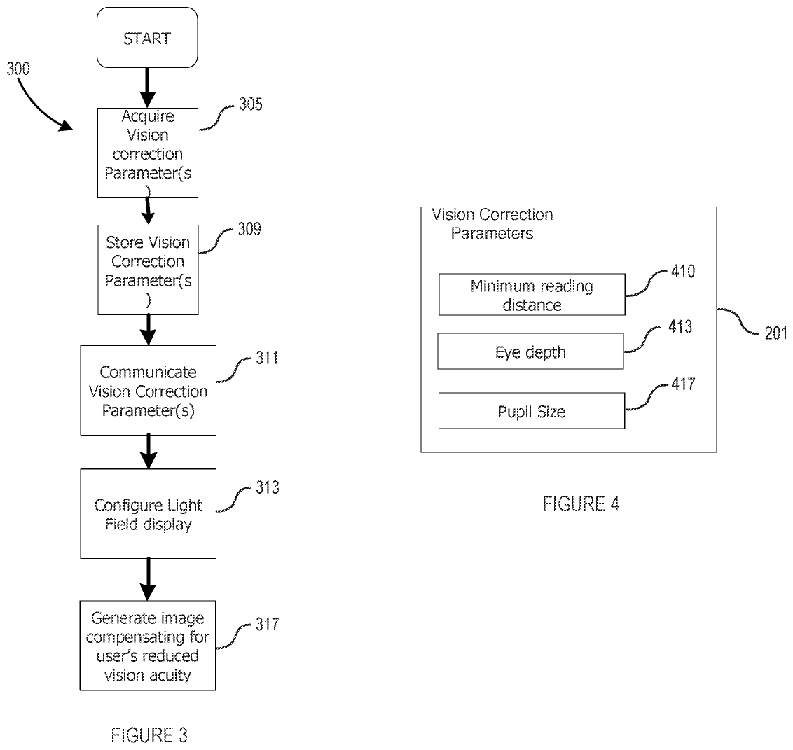

With reference to FIGS. 3 and 4, and in accordance with one exemplary embodiment, a vision correction management process using the vision correction system of FIG. 1 or 2, generally referred to using the numeral 300, will now be described. The first step in process 300 is to determine a set of vision correction parameter(s) 201 specifically tailored to the user's reduced visual acuity (step 305).

For example, FIG. 4 shows an exemplary set of vision correction parameters 201. These may include, without limitation, a user's corrective vision prescription value or parameters, such as a minimum reading distance 410 (or parameters(s) related thereto or representative thereof), first or higher order visual aberration characteristics or parameters, or the like. These parameters 201 may further include, but are not limited to, an optional eye depth 413 and/or pupil size 417 of the user, as may be utilized in different implementation of a corrective light field rendering process. In the illustrated embodiment, the minimum reading distance 410 is defined as the minimal focus distance for reading that the user's eye(s) may be able to accommodate (i.e. able to view without discomfort), and may be represented by a physical minimum reading distance, or again by an eyeglass prescription or corrective value, reading test result, or other like information as may be readily appreciated by the skilled artisan. In some embodiments, the system may use an average value of the eye depth 413 and/or pupil size 417 for all users. In some embodiments, this average may be changed as a function of physiological characteristics of the user (i.e. male or female, height, age, etc.). In some embodiments, other vision correction parameters may be considered depending on the application at hand and vision correction being addressed.

In some embodiments, the set of vision correction parameter(s) 201 may be derived from an eye prescription received from an optometrist, ophthalmologist or other certified eye-care professional. The eye prescription information may include the following data: left eye near spherical, right eye near spherical, left eye distant spherical, right eye distant spherical, left eye near cylindrical, right eye near cylindrical, left eye distant cylindrical, right eye distant cylindrical, left eye near axis, right eye near axis, left eye distant axis, right eye distant axis, left eye near prism, right eye near prism, left eye distant prism, right eye distant prism, left eye near base, right eye near base, left eye distant base, and right eye distant base. The eye prescription information may also include the date of the eye exam and the name of the eye doctor that performed the eye exam. The set of vision correction parameter(s) needed to compensate for the user's reduced visual acuity can be determined from this information, as will be explained below.

In some embodiments, the user may be provided directly with the set of vision correction parameter(s) by the eye care professional in a format corresponding to the exemplary list of FIG. 4 or similar.

In yet other embodiments, vision correction parameters may be dynamically acquired via the mobile device 202, for example, whereby a corrective light field image may be displayed and dynamically adjusted by the user via the mobile device 202 so to invoke and implement an onboard vision correction calibration process. For example, vision correction parameters may be automatically adjusted in response to a user action on the device 202 until an improved image rendering is confirmed by the user, vision correction parameters associated with such improved image rendering can then be stored and/or otherwise registered against the user's vision correction profile for future use. Such a dynamic onboard vision correction calibration process may include, but is not limited to, a slide or scroll bar adjustment interface, or like parameter adjustment interfaces allowing for a progressively adjustable image correction function to be applied to a calibration image rendered on the mobile device's light field display. These and other onboard calibration techniques may be self-guided, or again managed or assisted by a certified eye-care professional.

Going back to FIG. 3, once the set of vision correction parameter(s) 201 has been determined, at step 309 these may be entered or transferred into the memory or data storage 205 of a mobile digital device 202, as explained above. In some embodiments, data storage 205 may keep the set of vision correction parameter(s) in an encrypted form. The skilled technician will appreciate that any known encryption algorithm known in the art may be used, without limitation.

In some embodiments where the mobile device 202 further comprises a keyboard or graphical user interface (GUI), the set of vision correction parameter(s) 201 may be entered manually by the user (i.e. typed, etc.) into the mobile device 202. In some embodiments, the user may enter information regarding an eye prescription, as discussed above, and mobile device 202 may extract therefrom via processing unit 209 the corresponding set of vision correction parameter(s).

In some embodiments, mobile device 202 may not comprise any data entering mechanism nor any displays (e.g. a smart key or electronic fob). The set of vision correction parameter(s) 201 may then be pre-programmed or stored within mobile device 202 before being made available to the user, or again transferred thereto or encoded therein via a wired or wireless data transfer protocol, for example.

In some embodiments, as mentioned above, mobile device 202 may be a smartphone, tablet, laptop, or similar. In this case, system 100 may further include a dedicated mobile application and/or a website (not shown) accessible by mobile device 202 to control how the set of vision correction parameter(s) 201 is managed on the mobile device 202. In some embodiments, the set of vision correction parameter(s) 201 may be tied to a user profile or similar, which may contain additional user information such as a name, address or similar. The user profile may also contain additional medical information about the user. All information or data (i.e. set of vision correction parameter(s) 201, user profile data, etc.) may be kept on a remote database 231, and transferred to mobile device 202 via said mobile application or website. Similarly, in some embodiments, the user's current vision correction parameter(s) may be actively stored and accessed from an external database operated within the context of a server-based vision correction subscription system or the like, and/or unlocked for local access via the client application post user authentication with the server-based system. In some embodiments, the remote server or database may also record eye prescription data and extract therefrom the set of vision correction parameter(s) 201.

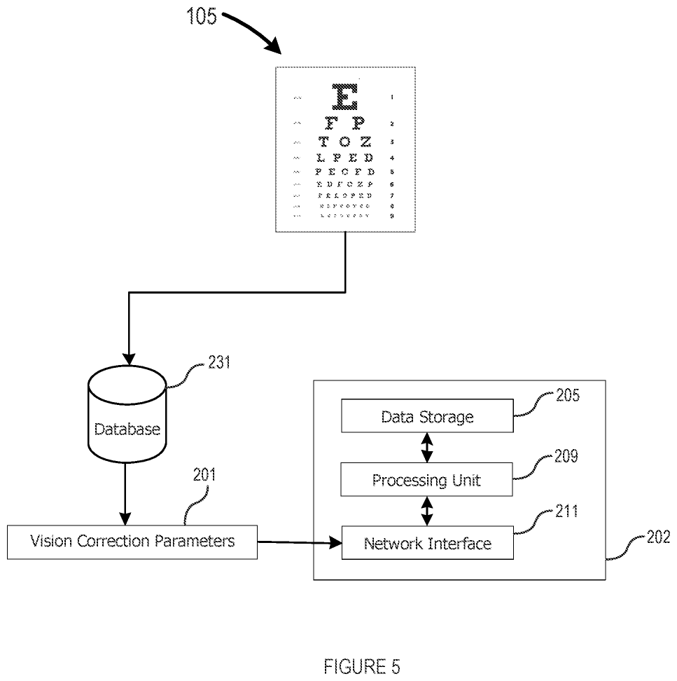

In some embodiments, as illustrated in FIG. 5, an optometrist or other eye-care professional may be able to transfer the user's eye prescription 105 directly and securely to his/her user profile store on said server or database. This may be done via a secure website, for example, so that the new prescription information is automatically uploaded to the secure user profile on remote database 231. Eye prescription 105 and/or a set of vision correction parameters 201 derived therefrom may be automatically uploaded to the user's mobile device 203 via network interface 211 of mobile device 202. In some embodiments, as above, the mobile application may be routinely or periodically synchronized with a remote server 231 (e.g. via a wireless data network) so to maintain up do date prescription metrics and data, and backup the user's local profile, for example. In some embodiments, the mobile application may be operable to locally extract and/or derive the set of vision correction parameter(s) corresponding to the prescription locally on mobile digital device 203. In some embodiments, the set of vision correction parameter(s) 201 may be stored in plain text format and/or in binary form.

In some embodiments, the system may comprise more than one set of vision correction parameter(s), e.g. each for a different user. For example, if the digital mobile device permits multiple accounts (i.e. via a mobile application on a smartphone or tablet, etc.), then multiple users may use the same mobile device but with their own personal accounts or profiles upon identification and/or authentication. For example, distinct preset or designated user accounts may be stored or accessed via a same mobile device so to allow such users to execute and distribute corrective light field display functions using this same device. For example, a given user of a particular multi-user device 202 may actively login, authenticate or otherwise access their profile so to actively communicate vision correction parameters associated therewith to a destination device. In the context of a vehicular interface, for example, distinct user profiles may be associated with a common key or fob such that different drivers/users may use this common key or fob to access vision correction functions for a shared vehicle.

In some embodiment, one or more features disclosed above of the dedicated mobile application may be integrated into the mobile device operating system or similar.

In some embodiments, one or more previous sets of vision correction parameter(s) 201 may be kept in memory 205 for reference and/or may be deleted upon entering a new set of vision correction parameter(s) 201.

In some embodiments, the set of vision correction parameter(s) 201 may in fact be multiple sets of vision correction parameter(s), each for a different user. For example, a user may be able to use mobile device 202 to choose which set of vision correction parameter(s) is to be used at a given moment.

In some embodiments, mobile device 202 may be operable to warn the user that the last received eye prescription information from which the current set of vision correction parameter(s) 201 is derived is now or will soon be out of date. In some embodiments, this warning may be communicated by said dedicated application, for example via a push notification or similar, or by said website via an email or text message, for example.

Going back to FIG. 3, once mobile device 202 has the set of vision correction parameter(s) 201 stored in data storage 205, the user may carry mobile device 202 on his/her person or in proximity thereto. At any moment where the user wants to interact with a device 203 equipped with a light field display 223, at step 335, the vision correction parameter(s) 201 may be automatically transmitted/communicated via network interface 211 of mobile device 202 through the use of a wired or wireless network connection to a corresponding receiving network interface 215 of device 203. The skilled artisan will understand that a different means of wirelessly connecting electronic devices may be considered herein, such as, but not limited to, Wi-Fi, Bluetooth, NFC, Cellular, 2G, 3G, 4G, 5G or similar. In some embodiments, in the case of a smartphone, tablet, e-reader or similar, the connection may be made via a connector cable (e.g. USB including microUSB, USB-C; Lightning connector; etc.). Once the set of vision correction parameter(s) 201 are stored in internal memory or storage unit 219, at step 313, processing unit 217 of device 203 may be operable to retrieve this set of vision correction parameter(s) 201 as needed to calibrate/configure a light field rendering process or similar at step 315, so to operate light field display 223 to render an image that, at least partially, compensates for the user's reduced visual acuity.

Generally, the set of vision correction parameter(s) 201, once received by device 203, may be stored, in some case only temporarily, within a data storage unit or internal memory 219. In some embodiments, the set of vision correction parameter(s) 201 may be stored in an encrypted form in data storage 219. In some embodiments, the set of vision correction parameter(s) 201 may be destroyed, removed or erased from internal memory 219 upon meeting a pre-determined condition, for example a time limit, when device 203 is turned off or moves out of range of device 203, or again when another user interacts with device 203.

In some embodiments, the system may require an additional identification and/or authentication step before configuring the light field display. These may include entering a password into the mobile device and/or any biometric identification/authentication methods known in the art.

In some embodiments, device 203 may monitor the user interacting with light field display 223. For example, device 203 may record the time of the user has been interacting with display 223 and send back this information to mobile device 202 or remote database 231 for statistical purposes and/or further adjustments to the set of vision correction parameters 201, as will be explained below.

In some embodiments, the set of vision correction parameter(s) 201 may be determined and/or updated via light-field capable display 223 of device 203 via a GUI or similar. In some embodiments, if the automatic transmission of the vision correction parameter(s) 201 failed, the user may be able to manually set his/her vision correction parameter(s) 201 via dials/buttons or via a graphical user interface or similar. Other methods may include real-time calibration without a prescription based on a few instant tests controlled by a user on light field display 223. In some embodiments, the updated set of vision correction parameter(s) may be transmitted back to mobile device 202 to be updated in memory 205.

As described above, various approaches, contexts and applications may be applied in accordance with different embodiments as described herein. Notably, while vision acuity parameters may be entered, transferred or relayed by the user and/or an eye care professional as determined via external means (e.g. standard eye or reading tests), onboard or system-initiated vision testing may also be implemented as a starting point, whether such online tests be initiated or established via the user's mobile device (and integrated mobile light field display), at a participating light field display kiosk, workstation or terminal, or again via a specialized vision testing light field display terminal or device operated or assisted by a vision or eye care specialists or technician.

Once vision acuity information is encoded by and/or for the system, it may then be (securely) stored in the user's mobile device and/or system database, to be subsequently relayed or transferred to participating light field display-enabled devices, terminals, kiosks, workstations, vehicles, etc., be it in the form of a dedicated or shared wireless (e.g. Bluetooth, NFC) pairing or communication stream, secure wireless vision correction profile token exchange or transfer, user vision profile identification and/or authentication protocol, or the like. Other wireless data transfers may also be considered, without departing from the general scope and nature of the present disclosure.

With reference again to FIG. 2, the system 200 may, in some embodiments, be further configured to manage or adapt to different time-based and/or contextual evolutions of the set of vision correction parameter(s) 201 (e.g. invoke a variable corrective power). Such variable parameter sets may, in some embodiments, transfer to corresponding light field display enabled devices 203, as described above, and/or alternatively, benefit the user for onboard vision correction, for example, where the mobile device 202 itself comprises a light field display. Namely, a variable user vision correction profile and/or parameters may be invoked both natively within the context of a self-contained vision correction apparatus or application, but also within the context of the distributed vision adaptation system described above.

In some embodiments, the set of one or more vision correction parameter(s) 201 may in fact be one or more sets of one or more vision correction parameter(s) associated with the same user. For example, each set may be specifically chosen to address known or expected changes in the user's reduced visual acuity over a range of viewing conditions, such as time-of-day, day vs. night vision, prolonged viewing time, ambient luminosity, display luminosity, rendered image detail levels, etc. Such sets may comprise different discretely set parameters, or again graduated parameters varying as per a corresponding scale, range or like variables.

Alternatively, system 200 may provide means to tune or modify an already existing set of vision correction parameter(s) 201 to compensate for new or changing viewing conditions, which changes can then be tracked, monitored and optionally automatically learned from over time to automatically mirror the user's viewing preferences. For example, in some embodiments, the user may interact with a display to manually change the corrective power (e.g. the set of vision correction parameters) to compensate for changing viewing conditions. The display would then store and track the new corrective power as a function of the current viewing conditions (time, viewing time, light level, etc.) and automatically change the corrective power of the display upon known viewing conditions being encountered once more. By introducing artificial intelligence metrics and learning capabilities, the device or system can then progressively adapt to previously encountered and new derivable viewing conditions to adapt the user's profile parameters accordingly. Various viewer demographics may also be taken into account, such as age, race, sex, viewing habits, occupation, etc., to further customize parameter predictability and time-varying or condition-varying operability.

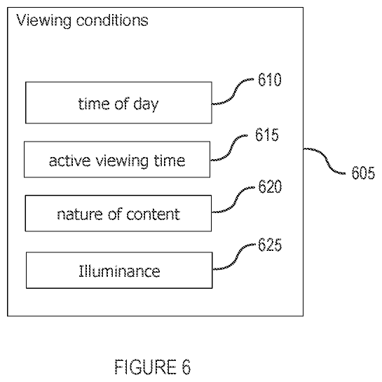

For example, as illustrated in FIG. 6, viewing conditions 605 may include, without limitation, the time of day 610, the active viewing time 615, the nature of the content viewed 620 and/or illuminance level 625 (e.g. ambient, display and/or relative illumance/luminosity). The time of day 610 can be measured using a digital clock by mobile device 202, device 203 or external database 231, and can invoke vision correction changes based on various discrete time ranges (e.g. morning, afternoon and/or nighttime), or again based on one or more awake time metrics such as to infer predictable or observed fatigue levels. This may be useful, for example, if users reportedly or typically experience further visual acuity degradation later in the day vs. in the morning, or again after being awake (e.g. after first use of the mobile device 202) for a number of hours, for example. In some embodiments, it may also include any information related to the day/night cycle.

Active viewing time 615 may include the total time the user has passed interacting with a given light field display in a single session (i.e. without a break) and/or during multiple sessions in one day. In some embodiments, a given number of minutes or hours may have to pass without any interaction before this total viewing time is reset to zero, or again, may decrease gradually based on one or more reported or observed variations. This information may again be recorded using a digital clock by device 203 and sent back to mobile device 202 or remote server 231, or again monitored directly by the mobile device 202 for onboard or distributed vision correction.

The nature of content viewed 620 may include, for example, whether the user is viewing text or images, in which case the user might need a stronger corrective effect when viewing text, or again when reading for prolonged periods as compared to consuming video and/or images that may be less fatiguing to the user than reading extensively. In some embodiments, the information about the different types of content included in an image may be already included into the digital image file being rendered by the light field display, or it may be detected in real-time by the rendering software executed by processing unit 217 on device 203 (or natively on the user's mobile device 202). In some embodiments, gaze tracking techniques or similar may also or alternatively be used to identify what type of content the user is watching as a function of time.

For example, cyclical left-to-right gaze tracking may automatically identify that the user is reading, and thus, apply an appropriate corrective power accordingly. In other examples, gaze tracking may help track viewer resting periods, e.g. where a user routinely looks away from the display to adjust their focus and thus rest their eyes intermittently during viewing. Such best practices may be observed and/or encouraged by the device, and ultimately used to adjust the displays corrective power.

Illuminance 625 may comprise the ambient light level or illuminance above or in the vicinity of the user or display, or again include a brightness or relative brightness of the display itself. For example, system 200 may use information from additional sensors (not shown) equipped either on the user's mobile device 202 or on the light field display equipped device 203 or in proximity to either, to detect changes in ambient lighting levels which, for example, may not be time-of-day related. For example, a user driving or being a passenger on a vehicle entering a tunnel, or naturally or artificially enclosed area, or again driving in changing environmental conditions (sunny vs. overcast) may require an increased corrective power; system 200 may therefore automatically detect the change in light level and automatically communicate/transmit an adjusted set of vision correction parameter(s) 201 to the onboard light field display 223 to compensate for the user's currently reduced visual acuity.

Other vision-impacting parameters and conditions may also apply within the present context, without departing from the general scope and nature of the present disclosure.

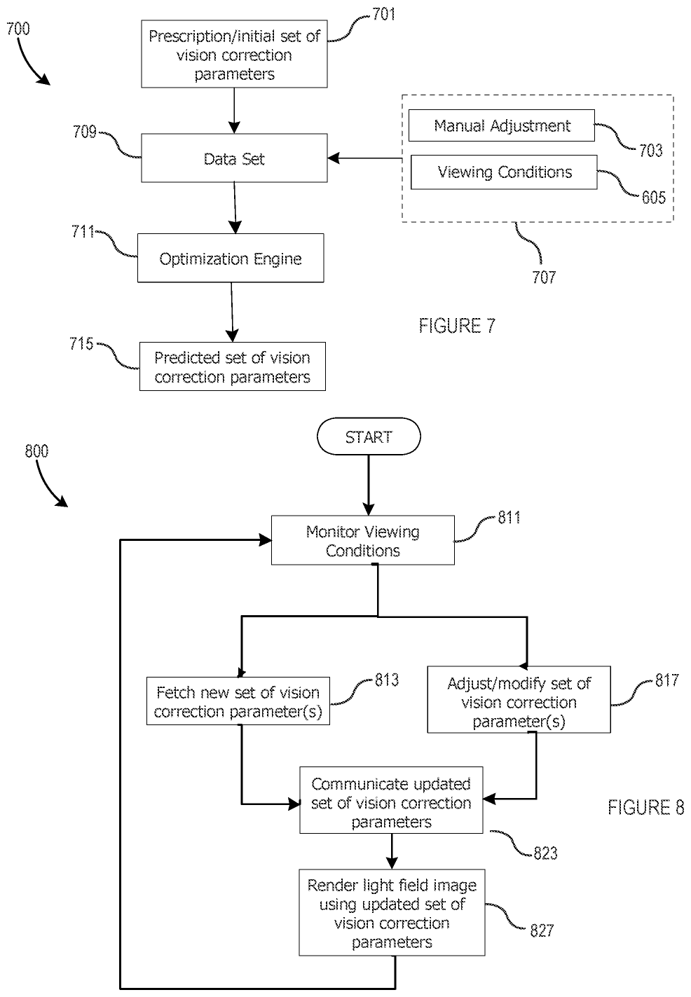

With reference to FIG. 7, and in accordance with one exemplary embodiment, a dynamic corrective power management system for light field displays, generally referred to using the numeral 700, will now be described. In some embodiments, system 700 may take as initial input an initial set of vision correction parameters (or a prescription form which they may be derived) 701. In some embodiments, the user may make subsequent manual adjustments 703 to the currently used set of vision correction parameters due to a change in one or more viewing condition 605. Accordingly, a given device 203 may be adapted to compensate for different visual acuity levels and thus accommodate different users and/or uses. For instance, a particular device may be configured to implement and/or render an interactive graphical user interface (GUI) that incorporates a dynamic vision correction scaling function that dynamically adjusts one or more designated vision correction parameter(s) in real-time in response to a designated user interaction therewith via the GUI. Such dynamic controls may be presented to adjust input corrective parameters, or again, to initiate a first set of parameters through onboard calibration. For example, a dynamic vision correction scaling function may comprise a graphically rendered scaling function controlled by a (continuous or discrete) user slide motion or like operation, whereby the GUI can be configured to capture and translate a user's given slide motion operation to a corresponding adjustment to the designated vision correction parameter(s) scalable with a degree of the user's given slide motion operation. These and other examples are described in Applicant's co-pending U.S. patent application Ser. No. 15/246,255, the entire contents of which are hereby incorporated herein by reference.

As illustrated in FIG. 7, in some embodiments, an initial prescription and/or initial set of vision correction parameters 701 may be recorded into a dataset 709. Dataset 709 may further be augmented with any subsequent manual adjustment 701 and it's associated viewing conditions 705 as a data point 707. An optimization engine 711 may use any or all data in database 707 to subsequently automatically generate therefrom a plurality of optimized sets of vision correction parameters 715 adapted specifically for one or more viewing conditions. In some embodiments, optimization engine 711 using the initial set of vision correction parameters and current viewing conditions as input may be used by the mobile device 202 and/or device or system 203 and/or remote server 231 to produce/generate a newly updated/adapted set of vision correction parameters. For example, optimization engine 711 may use a linear or non-linear model using different weight functions for each viewing condition.

In some embodiments, optimization engine 711 may use an artificial intelligence system to provide automated or semi-automated optimization strategies, such as for example, generating from a first input (e.g. an eye prescription) and/or subsequent user manual adjustments 701 the best set of vision correction parameters for a range of viewing conditions. Different AI, machine learning and/or system automation techniques may be considered. For example, these may include, without limitation, supervised and/or unsupervised machine learning techniques, linear and/or non-linear regression, decision trees, etc. Deep learning algorithms may also be used, including but not limited to, neural networks such as recurrent neural networks, recursive neural networks, feed-forward neural networks, convolutional neural networks, deep-belief networks, multi-layer perceptrons, self-organizing maps, deep Boltzmann machines, and stacked de-noising auto-encoders or similar. As such, the optimization engine 711 is designed to operate autonomously or semi-autonomously, with limited or without explicit user intervention. Every time the user manually changes the corrective power of the display, this information is added to the optimization engine 711 to include that modification to train itself to provide more accurate sets of vision correction parameters in the future.

In some embodiments, dataset 709 may be contained on data storage or internal memory 205 of mobile device 202, internal memory 219 of system 203 and/or remotely on remote database 231. In some embodiments, dataset 709 may not have to be processed on the same device/system; it may be transmitted/communicated, at least partially, to an external processing unit.

With reference to FIG. 8, and in accordance with one exemplary embodiment, a time-variable corrective light field display management method using the system of FIG. 7, generally referred to using the numeral 800, will now be described. In some embodiments, before the user first starts interacting with device 203, at step 811, a check or monitoring is made to any or all viewing conditions 605, as discussed above. As explained, some of these may require the use of additional sensors, such as a light sensor for example, either on device 203 and/or mobile device 202, while others only need a digital clock. Therefrom, method 800 may either, at step 813, fetch an adjusted set of vision correction parameters 201 that has been recorded from a previous user input when the current viewing conditions 605 were last encountered (e.g. data point 707 in FIG. 7); or it may instead, at step 817, use optimization engine 711 to generate a predicted set of vision correction parameters 715 to better compensate for the user's reduced visual acuity in the current viewing conditions. This may be useful if the current viewing conditions 605 are close but not identical to those used to record a previously recorded data point 707.

Once identified or generated, the new set of vision correction parameter(s) may be transmitted/communicated, at step 823, to device 203 where they are used to calibrate light field rendering algorithm to render at step 827 an updated image. The method goes back to step 811 to continue monitoring for any change in one or more viewing condition 605. In some embodiments, this monitoring may be done continuously or in others, periodically. In some embodiments, steps 811 to 825 may be done only once before the user starts interacting with the light field display. In some embodiments, different means of getting new sets of vision correction parameters may be offered to the user via a user interface, notification system or similar (for example using either step 813 or 817, or using different machine learning models in optimization engine 711 at step 817).

In some embodiments, the optimization engine may also monitor the user's viewing habits in addition to the viewing conditions 605. A viewing habit is the combination of time, location, close proximity to other users or other contextual data associated with one or more interaction with a digital display. Optimization engine 711 may use viewing habits data to further increase its predictive power.

In some embodiments, a distinct optimization engine 711 may be implemented individually into each device or system 203 and work independently of mobile device 202 or server 231. Thus, step 823 may be omitted as steps 811 to 817 are done directly on device or system 203 and no wired or wireless communication is necessary.

### Example 1: Vehicular Implementation

One example of an environment wherein systems and methods as described herein may be used is in a vehicular context (e.g. the user is either driving or being a passenger in car, truck or similar). In the case where the mobile device is a smartphone, tablet or similar, the configuration/calibration of a light field capable display may be done via Bluetooth pairing, for example, or again other NFC or like short distance communication protocols. After an initial authentication and/or configuration step, the user's vision correction parameter(s) can be sent to an onboard vehicular data management system so to be applied to any one or more displays in the vehicle, such as a dashboard operating and/or multimedia display. This information can be securely stored on the vehicular data management system for future access, or again transfer thereto upon subsequent pairings between the phone and the vehicle. In other embodiments, the system may interact with a vehicle's dashboard display or other displays via a standard or API such as Apple CarPlay™, Android Auto™, or any other infotainment system or similar. In this example, an application would be accessible on the dashboard display via an icon or similar, from which the user may further configure/change the parameters of the dashboard display or any other display in the vehicle to use his/her designated vision correction parameter(s).

In some embodiments where the system is meant to be used exclusively in a vehicular setting, an electronic key, smart key or electronic key fob may be used as the personal mobile device storing and communicating the user's vision correction parameter(s). Thus, the vehicle may be operable to automatically configure any light field display within the vehicle to use the user's vision correction parameter(s). Continuing with the example of a vehicular environment, in some embodiments, the vision correction parameter(s) may be used to adjust other non-display related parameters. For example, the pupil distance to the screen may otherwise or additionally be rather approximated or adjusted based on other contextual or environmental parameters, such as an average or preset user distance to the screen via a set or adjustable seat position, etc.

In some embodiments, multiple users sitting inside a vehicle may each have one or more displays configured with their corresponding vision correction parameter(s). The system may be operable to detect or be configured to know which seat a user is sitting on to automatically configure/calibrate any light field display corresponding to that seating position. For example, a user driving the vehicle and a passenger sitting in the back seat may have the vehicle's dashboard display and an embedded multimedia display configured for each user respectively. This may be implemented, for example, and managed via an onboard seat/multimedia allocation management system, or again, provided through respective seat-specific data management pairings or transfers, for example, where each seat in the vehicle is provided with a corresponding pairing receiver or ID.

Continuing with this example, seat-specific pairings may be adapted for public or commercial transportation embodiments whereby each user may actively pair their mobile device with an onboard allocated display or the like, such at those provided in the back, head or armrests of airplane seats, or the like. Given the public nature of such transportation vehicles, user vision correction identification and/or parameters may be securely transferred and temporarily stored during use, and automatically erased once a particular use is terminated, or again, when an onboard entertainment system is reset (e.g. for a new flight or travel destination).

Once again, where a variable corrective profile is implemented, such viewing condition variables may be applied, for example, due to changing ambient lighting conditions, time-of-day, viewing duration, etc.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

1. A digital vision correction system to at least partially address a user's reduced visual acuity, the system comprising:
a user mobile device comprising:
a processing unit;
a digital data storage to store a digital vision correction parameter associated with the user's reduced visual acuity; and
a wireless network interface; and
a distinct electronic display device comprising:
a light field display operable to render digital content;
a network interface operable to interface with said user mobile device to access said vision correction parameter; and
a processing unit, communicatively linked to said light field display and network interface, and operable on pixel data associated with said digital content to adjust a rendering thereof via said light field display as a function of said digital vision correction parameter so to at least partially address the user's reduced visual acuity.

2. The system of claim 1, wherein the system comprises a plurality said distinct electronic display device, each operable to respectively interface with said user mobile device to access said digital vision correction parameter and thereby output vision-corrected digital content.

3. The system of claim 2, wherein each said distinct electronic display device is further operable to automatically delete said vision correction parameter therefrom upon termination of a given user's interaction therewith, and access a distinct vision correction parameter for a distinct user upon interfacing with a distinct user mobile device.

4. The system of claim 1, wherein said distinct electronic display device comprises an onboard vehicular data processing device, and wherein said vision correction parameter is accessed from said user mobile device upon wirelessly pairing said user mobile device with said onboard vehicular data processing device.

5. The system of claim 1, wherein said distinct electronic display device comprises an electronic kiosk, and wherein said vision correction parameter is accessed from said user mobile device upon wirelessly interfacing said user mobile device with said electronic kiosk.

6. The system of claim 1, wherein said communication interface comprises at least one of a Bluetooth™ interface or a Near Field Communication (NFC) interface.

7. The system of claim 1, wherein said vision correction parameter is entered or derived from a manual user input.

8. The system of claim 1, wherein said vision correction parameter is entered or derived from a network-interfacing connection to an eye care specialist terminal.

9. The system of claim 1, wherein said user mobile device comprises a light field enabled display operable to render vision-corrected digital content, and wherein said vision correction parameter is dynamically adjusted via a graphical interface rendered on said user mobile device.

10. The system of claim 1, wherein said user mobile device comprises one of a mobile communication device, an electronic key, an electronic key fob, a digital identification card, and/or a wearable device.

11. A digitally implemented vision correction method for adjusting digital content to be rendered by an electronic display device in accordance with distinct digital user profiles each having a respective reduced visual acuity associated therewith, the method comprising, for each given digital user profile:
wirelessly interfacing the electronic display device having a light field display with a user mobile device to provide wireless access thereto to a digital user-specific vision correction parameter stored thereon or in association therewith and associated with the respective reduced visual acuity;
processing pixel data associated with the digital content to adjust a rendering thereof via said light field display as a function of said digital vision correction parameter; and
rendering said adjusted pixel data via said light field display to at partially address the respective reduced visual acuity.

12. The method of claim 11, further comprising digitally storing said digital user-specific vision correction parameter on said user mobile device via a communication interface between said user mobile device and an eye care specialist system.

13. The method of claim 11, further comprising digitally defining said digital user-specific vision correction parameter on said user mobile device via an onboard vision correction adjustment application executed on said mobile device to be interactively operated by the user in setting said digital user-specific vision correction parameter.

14. The method of claim 11, further comprising digitally storing said digital user-specific vision correction parameter on said user mobile device via a communication interface between said user mobile device and a distinct digital vision correction testing device operating a vision correction adjustment application thereon to be interactively operated by the user in setting said digital user-specific vision correction parameter.

15. The method of claim 11, wherein said wirelessly interfacing comprises automatically pairing or wirelessly interfacing said user mobile device upon said user mobile device being in the vicinity of said electronic display device.

16. The method of claim 11, wherein said user mobile device comprises a smartphone, a tablet, a laptop, a smart key, a key fob or an identification card.

17. The method of claim 11, wherein said wirelessly interfacing comprises securely transferring said digital user-specific vision correction parameter.

18. The method of claim 17, wherein said digital user-specific vision correction parameter is securely transferred via a secure digital token.

19. The method of claim 11, further comprising, upon termination of said interfacing, deleting said user-specific vision correction parameter from said electronic display device.

20-36. (canceled)
